(19) United States
(12) Patent Application Publication
(10) Pub. No.: US 2024/0417662 A1
(43) Pub. Date: Dec. 19, 2024

(54) CELL DETACHMENT SYSTEM AND CELL CULTURE SYSTEM

(71) Applicants: CANON KABUSHIKI KAISHA, Tokyo (JP); CANON MEDICAL SYSTEMS CORPORATION, Tochigi (JP)

(72) Inventors: HISAFUMI EBISAWA, Tokyo (JP); YOJI YAMAMOTO, Tokyo (JP); ICHIRO HARADA, Kanagawa (JP)

(21) Appl. No.: 18/731,561

(22) Filed: Jun. 3, 2024

(30) Foreign Application Priority Data
Jun. 14, 2023 (JP) 2023-098040

(51) Int. Cl. C12M 1/26; C12M 1/00; C12M 1/36

(52) U.S. Cl. CPC C12M 33/18; C12M 23/50; C12M 41/48

(57) ABSTRACT

Provided is a cell detachment system including: a scraper having magnetism, for detaching cells adhering to a culture surface; a magnetic field applying unit configured to apply a magnetic field to the scraper; and a scraper position control unit configured to control a position of the scraper by controlling the magnetic field applying unit, wherein the scraper is configured to move while rotating under a state in which no magnetic field is applied thereto, and under a state in which a magnetic field is applied thereto by the magnetic field applying unit, to be caused to move, without rotating, by the scraper position control unit so as to detach the adhering cells.

### BACKGROUND OF THE INVENTION

### Field of the Invention

The present invention relates to a cell detachment system and a cell culture system.

### Description of the Related Art

In culturing of adherent cells, there is a step called cell detachment. In general, at a time of detachment, there is adopted an approach of weakening intercellular adhesion by enzymes and then physically detaching cells through use of a scraper or the like. Further, cell culture is required to be performed in a clean area to prevent bacteria and other sample cells from entering. In actuality, there have been developed a large number of systems in which, in a clean area, cells are seeded in a culture vessel and a culture medium is supplied thereto and drained therefrom. To detach cells in such a system, it is required to insert a scraper into a container and scrape a culture surface. However, the cells, the scraper, and a drive system that holds and operates the scraper are present in the same space, thereby requiring preparation of a clean area even for the drive system. This is a factor that hinders cost reduction.

In order to solve this problem, as in Japanese Patent Application Laid-Open No. 2004-129558, there has been developed a system in which a triangular prism scraper made of a magnetic substance is installed in a closed container and a magnetic force is used to drive the scraper from outside the closed container, to thereby detach cells by the scraper while keeping the inside of the container clean.

In Japanese Patent Application Laid-Open No. 2004-129558, a simple culture system in which only one scraper having a triangular prism shape is installed in a culture vessel is achieved. However, there is a fear that, when a scraper is merely installed in a culture vessel, the scraper may come into contact with cells being cultured to detach the cells. For example, in Japanese Patent Application Laid-Open No. 2004-129558, the scraper is not fixed in a container in a normal state, and it is conceivable that the scraper moves in the container due to a water flow or tilting of the container at a time of culture medium replacement and cells being cultured are detached by a culture surface contact portion of the scraper while the scraper is moving.

### SUMMARY OF THE INVENTION

The present invention has been made in view of the above-mentioned problem. An object of the present invention is to provide a cell detachment system in which a scraper is prevented from coming into contact with cells, or even when the scraper comes into contact with the cells, friction is suppressed during cell culture, thereby reducing damage caused to the cells at a time of culture and also enabling the scraper to detach the cells while a closed system is maintained.

The present invention adopts the following configurations. That is, there is provided a cell detachment system including: a scraper having magnetism, for detaching cells adhering to a culture surface; a magnetic field applying unit configured to apply a magnetic field to the scraper; and a scraper position control unit configured to control a position of the scraper by controlling the magnetic field applying unit, wherein the scraper is configured to move while rotating under a state in which no magnetic field is applied thereto, and under a state in which a magnetic field is applied thereto by the magnetic field applying unit, to be caused to move, without rotating, by the scraper position control unit so as to detach the adhering cells.

In addition, according to one aspect of the present invention, there is provided a cell culture system including: the above-mentioned cell detachment system; and a culture vessel including the culture surface.

In addition, according to one aspect of the present invention, there is provided a cell detachment system including: a scraper having magnetism, for detaching cells adhering to a culture surface; a magnetic field applying unit configured to apply a magnetic field to the scraper; a scraper position control unit configured to control a position of the scraper by controlling the magnetic field applying unit; and a scraper holding portion configured to hold the scraper at a position out of contact with the culture surface.

In addition, according to one aspect of the present invention, there is provided a cell culture system including: the above-mentioned cell detachment system; and a culture vessel including the culture surface.

Further features of the present invention will become apparent from the following description of exemplary embodiments with reference to the attached drawings.

### BRIEF DESCRIPTION OF THE DRAWINGS

FIG. 1A is a perspective view for illustrating an outline of an overall configuration of an example of a cell culture system including a cell detachment system according to the present invention, and FIG. 1B is a perspective view for illustrating a state in which cells adhering to a culture surface are being detached by a scraper in the example of the cell culture system illustrated in FIG. 1A.

FIG. 2 is a perspective view for illustrating how spherical scrapers in the present invention are installed on a culture surface having recessed curved surfaces.

FIG. 3A, FIG. 3B, and FIG. 3C are views for each illustrating an example of a columnar or cylindrical scraper in the present invention, in which FIG. 3A is a perspective view for illustrating a columnar scraper made of a magnetic substance, FIG. 3B is a perspective view for illustrating a columnar scraper having a magnetic substance used in a central part and a non-magnetic substance in a peripheral part, and FIG. 3C is a perspective view for illustrating a cylindrical scraper made of a magnetic substance.

FIG. 4A is a schematic view for illustrating how a related-art scraper is moving due to an external force while coming into contact with cells, FIG. 4B is a schematic view for illustrating how the scraper in the present invention is moving due to an external force while coming into contact with cells, and FIG. 4C is a schematic view for illustrating how the scraper in the present invention is detaching cells.

FIG. 5A, FIG. 5B, FIG. 5C, and FIG. 5D are schematic views for each illustrating an example of the cell detachment system according to the present invention in which a scraper holding portion is installed in a culture vessel, in which FIG. 5A is a view for illustrating a state during cell culture in a cell detachment system in which the scraper holding portion has a slope, FIG. 5B is a view for illustrating how to move the scraper to a culture surface in the cell detachment system after cell culture, FIG. 5C is a view for illustrating a state during cell culture in a cell detachment system in which the scraper is held by being attracted by a magnet, and FIG. 5D is a view for illustrating how to move the scraper to the culture surface in the cell detachment system after cell culture.

FIG. 6A and FIG. 6B are views for each illustrating an example of the cell detachment system according to the present invention in which a scraper holding portion is installed outside a culture vessel, in which FIG. 6A is a schematic view for illustrating a state during cell culture, and FIG. 6B is a schematic view for illustrating how to move the scraper to a culture surface after cell culture.

FIG. 7 is a schematic view for illustrating an example of an overall configuration of a cell culture system including a cell detachment system according to a first embodiment.

FIG. 8A, FIG. 8B, and FIG. 8C are schematic views for each illustrating an example of an overall configuration of a cell culture system including a cell detachment system according to a second embodiment, in which FIG. 8A is a view for illustrating a state exhibited before a scraper is moved to a culture surface, FIG. 8B is a view for illustrating how to move the scraper to the culture surface, and FIG. 8C is a view for illustrating a state in which the movement of the scraper to the culture surface has been completed.

FIG. 9A, FIG. 9B, and FIG. 9C are schematic views for each illustrating an example of an overall configuration of a cell culture system including a cell detachment system according to the second embodiment, in which FIG. 9A is a view for illustrating a state exhibited before a scraper is moved to a culture surface, FIG. 9B is a view for illustrating how to move the scraper to the culture surface, and FIG. 9C is a view for illustrating a state in which the movement of the scraper to the culture surface has been completed.

FIG. 10A, FIG. 10B, and FIG. 10C are schematic views for each illustrating an example of an overall configuration of a cell culture system including a cell detachment system according to a third embodiment, in which FIG. 10A is a view for illustrating a state exhibited before a scraper is moved to a culture surface, FIG. 10B is a view for illustrating how to move the scraper to the culture surface, and FIG. 10C is a view for illustrating a state in which the movement of the scraper to the culture surface has been completed.

FIG. 11A, FIG. 11B, and FIG. 11C are schematic views for each illustrating an example of an overall configuration of a cell culture system including a cell detachment system according to a fourth embodiment, where FIG. 11A is a view for illustrating a state exhibited before scrapers are moved to a culture surface, FIG. 11B is a view for illustrating how to move the scrapers to the culture surface, and FIG. 11C is a view for illustrating a state in which the movement of the scrapers to the culture surface has been completed.

FIG. 12 is a block diagram for illustrating a configuration example of the cell detachment system according to the present invention.

FIG. 13 is a block diagram for illustrating a hardware configuration example of an information processing unit in the present invention.

### DESCRIPTION OF THE EMBODIMENTS

A cell detachment system according to the present invention includes: a scraper having magnetism, for detaching cells adhering to a culture surface; a magnetic field applying unit configured to apply a magnetic field to the scraper; and a scraper position control unit configured to control a position of the scraper by controlling the magnetic field applying unit, wherein the scraper is configured to move while rotating under a state in which no magnetic field is applied thereto, and under a state in which a magnetic field is applied thereto by the magnetic field applying unit, to be caused to move, without rotating, by the scraper position control unit so as to detach the adhering cells.

Exemplary embodiments of the present invention are described below with reference to the accompanying drawings. In the figures, like components are denoted by like reference numerals in principle, and description thereof is omitted. The term “external force” as used in the present specification refers to a force other than a magnetic force, and examples thereof include gravity, an inertial force, and a water flow that are applied when a culture vessel is moved. The term “sample solutions and the like” as used in the present specification refers to a culture medium, an enzyme solution, phosphate-buffered saline (PBS), a cell suspension, and the like. In the following description, an adherent cell is described as an example, but a cell detachment system and a cell culture system according to the present invention can also be applied to cells other than adherent cells.

### (Configuration of System)

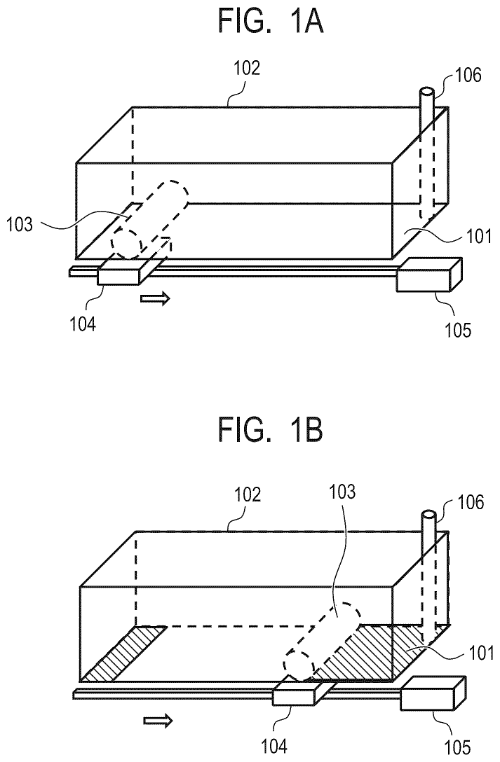

First, the cell detachment system and the cell culture system including the cell detachment system according to the present invention are described with reference to FIG. 1A and FIG. 1B. FIG. 1A is a perspective view for illustrating an outline of an overall configuration of an example of the cell culture system including the cell detachment system according to the present invention. A culture surface 101 has a surface to which cells can adhere and on which the cells can be proliferated. A culture vessel 102 includes the culture surface 101 and is capable of holding a liquid. A scraper 103 has magnetism, and is used for detaching cells adhering to the culture surface. The scraper 103 slides in a direction indicated by the arrow. A magnetic field applying unit 104 applies a magnetic field to the scraper. A scraper position control unit 105 controls the magnetic field applying unit to control a position of the scraper 103 in the culture vessel 102. The scraper 103 moves while rotating under a state in which no magnetic field is applied thereto. Under a state in which a magnetic field is applied thereto by the magnetic field applying unit 104, the scraper 103 is moved by the scraper position control unit 105 without rotating so as to detach the cells adhering to the culture surface 101.

On a wall surface of the culture vessel 102, a supply and drainage port 106 that is inserted in the culture vessel 102 so as to allow a culture medium and the like to be taken in and out therethrough is present. An upper surface of the culture vessel 102 is kept closed, and is connected to the outside through the supply and drainage port 106. A plurality of supply and drainage ports 106 can be provided, and are connected to respective tubes (not shown) to be connected to storage containers (not shown) of the sample solutions and the like, such as a culture medium and PBS, through the tubes under aseptic conditions. It is also possible to create a closed system by providing a wide opening in place of the upper surface of the culture vessel, injecting the sample solutions and the like from this wide opening, and then tightly closing a lid therefor. However, from the viewpoint of preventing foreign matter from entering from the outside during the injection, it is preferred to provide a supply and drainage port and cause the opening to be narrow as illustrated in FIG. 1A. It is possible to prepare an incubator (not shown) as a cell culture unit, and to install the culture vessel 102 therein.

FIG. 1B is a perspective view for illustrating a state in which the cells adhering to the culture surface 101 are being detached by the scraper 103 in the cell culture system including the cell detachment system according to the present invention. In FIG. 1B, the hatched portions on the culture surface 101 indicates that cells adhere thereto. The scraper 103 to which a magnetic field is applied by the magnetic field applying unit 104 is controlled by the scraper position control unit 105 to move on the culture surface 101 while sliding thereon, to thereby be able to detach the cells adhering to the culture surface 101.

### (Culture Vessel)

The cell culture system according to the present invention can include the cell detachment system according to the present invention and a culture vessel including the culture surface.

The culture surface is a culture substrate that enables cells to adhere thereto and proliferate thereon, and a transparent material that facilitates observation is preferred to be used for the culture surface. A transparent resin, glass, or the like that facilitates processing and observation is preferred as a material of the culture surface. In a case of using a resin, polystyrene, polycarbonate, acrylic, or the like is usable. The material is not limited to a transparent material as long as the material does not block an optical path at a time of observation, but a material having magnetism, such as some kinds of metal, is not appropriate. The culture surface may form a part or all of the culture vessel, or may be provided separately from the culture vessel. In terms of cost and ease of preparation, it is preferable that the culture surface is the same material as the culture vessel and that the culture surface forms a part of the culture vessel.

A surface treatment such as a plasma treatment is preferred to be applied to the culture surface so that cells are likely to adhere only to the culture surface.

The culture vessel is a container that holds a culture medium for culturing cells. A transparent resin, glass, or the like that facilitates processing and observation is preferred as a material of the culture vessel. In a case of using a resin, polystyrene, polycarbonate, acrylic, or the like is usable. The material is not limited to a transparent material as long as the material does not block an optical path at a time of observation, but a material having magnetism, such as some kinds of metal, is not appropriate. The culture vessel is preferred to be a closed system from the viewpoint of being able to prevent foreign matter from entering from the outside and ensure a predetermined temperature and a predetermined humidity.

### (Scraper)

The cell detachment system according to the present invention includes a scraper having magnetism, for detaching the cells adhering to the culture surface.

The scraper may be caused to move while rotating under the state in which no magnetic field is applied thereto, and under the state in which a magnetic field is applied thereto by the magnetic field applying unit, may be caused to move, without rotating, by the scraper position control unit so as to detach the adhering cells. The term “rotating” as used herein refers to rotation due to an external force, and includes not only continuous rotation but also intermittent rotation. Specifically, the term “rotating” includes moving while rotating by a fixed distance, then moving while sliding without rotating, and again moving while rotating. The scraper can move while rotating under the state in which no magnetic field is applied thereto, thereby enabling the scraper to rotate on the culture surface when an external force is applied to the culture vessel during cell culture. Accordingly, it is possible to suppress a frictional force exerted by the scraper on cells being cultured. In this case, it is possible to reduce damage to the cells being cultured, and achieve a simple cell detachment system that is low in cost. Further, the scraper can be caused to move, without rotating, by the scraper position control unit under the state in which a magnetic field is applied thereto by the magnetic field applying unit, thereby enabling a magnetic field to be applied to detach cells at a time of cell detachment.

A shape of the scraper is not limited as long as the scraper is rotatable on the culture surface, and examples thereof include polyhedral shapes such as a regular polyhedron and a truncated polyhedron, a spherical shape, an ellipsoidal shape, a columnar shape, and a cylindrical shape. Examples of the polyhedral shapes include a regular polyhedron having 20 or more faces and a truncated polyhedron thereof. Of those, the shape of the scraper is preferred to be a shape with a surface of the scraper to be brought into contact with the culture surface being a curved surface, or a shape with a surface of the scraper to be brought into contact with the culture surface being formed only of a curved surface, such as a spherical shape, an ellipsoidal shape, a columnar shape, and a cylindrical shape. In a case of using a scraper having such a shape, the scraper is assumed to be arranged so that the surface of the scraper being a curved surface is brought into contact with the culture surface. In the present invention, a case in which the scraper is columnar includes cases in which the scraper is a right circular cylinder and an elliptic cylinder. Further, in the present invention, in a case in which the scraper is an ellipsoidal shape, assuming that x-axis, y-axis, and z-axis lengths of an ellipsoid are represented by “a”, “b”, and “c”, respectively, the ellipsoid can be set such that a/b, b/c, and c/a are all equal to or larger than ⅓ and equal to or smaller than 3. In the present invention, in a case in which the scraper has an elliptic cylindrical shape, assuming that a ratio of a short diameter and a long diameter of an elliptical surface is set to (short diameter):(long diameter)=a:b and “a” is 1, the scraper can be set to have such a shape that “b” is larger than 1 and equal to or smaller than 3.

In a case in which the culture surface is a flat surface, for example, when the shape of the scraper is a columnar shape or a cylindrical shape, a contact region between the culture surface and the scraper is a straight line. At the time of cell detachment, the scraper is moved while maintaining this contact region, thereby being able to detach cells on the region of the culture surface through which the contact region have passed. The contact region of the scraper with respect to the culture surface is preferred to be a line or a plane.

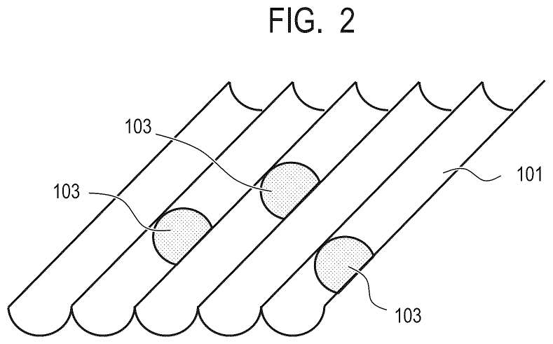

Further, when the culture surface in the present invention includes a recessed curved surface, the shape of the scraper is preferred to be a spherical shape or an ellipsoidal shape. The culture surface being a recessed curved surface substrate is advantageous in that the culture surface area per unit volume of the culture vessel can be increased more than in a case of the culture surface being a flat surface substrate. FIG. 2 is a perspective view for illustrating how spherical scrapers 103 in the present invention are installed on the culture surface 101 having recessed curved surfaces. When the culture surface is a recessed curved surface or a tubular surface, it is possible to use a scraper having such a spherical shape or an ellipsoidal shape as to conform to the recessed curved surface or the tubular surface. For example, when a recessed curved surface substrate having a curvature with a diameter of 1 mm is set as the culture surface, in a case of culturing cells on the culture surface, it is preferred to use a scraper that is a spherical body with a diameter of 1 mm or a scraper that is an ellipsoid with any one of a major axis, a minor axis, or a depth (x-axis, y-axis, or z-axis) being 1 mm.

The scraper contains a material having magnetism, and is preferred to contain, in particular, a metal having strong magnetism. A resin material or the like kneaded with magnetic particles can also be used. It is also effective to use different members to form a central part and a peripheral part of the scraper. For example, it is also effective to use a metal having magnetism as the central part and to cover, as the peripheral part, a cushioned rubber member having no magnetism. Covering with the rubber member allows use of a magnetic metal that is prone to corrosion as well, and cushioning properties thereof increase adhesiveness of the scraper at a time of detachment, thereby being able to improve detaching performance. In addition, a hollow scraper can be used. A case in which the scraper in the present invention has a cylindrical shape includes a case in which the scraper is a right circular cylinder or an elliptic cylinder that is hollow. A case in which the shape of the scraper is a spherical shape or an ellipsoidal shape includes a case in which the scraper is a hollow spherical shape or a hollow ellipsoidal shape. Through use of a hollow scraper, the detached cells and liquid pushed out at the time of cell detachment enters a hollow space, and the scraper can be smoothly moved without a path thereof being obstructed. Further, the scraper can be reduced in weight, and it is possible to further reduce damage to the cells being cultured when the scraper comes into contact with those cells.

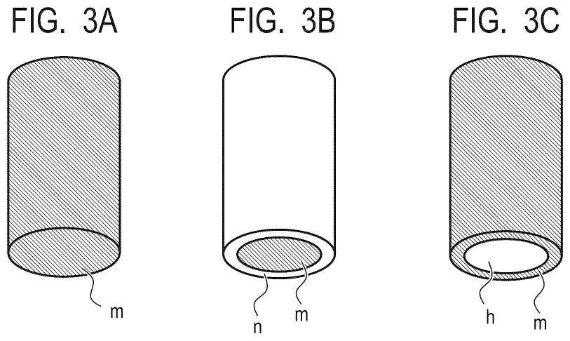

In FIG. 3A to FIG. 3C, examples of the columnar or cylindrical scraper are illustrated. FIG. 3A is a perspective view for illustrating a columnar scraper made of a magnetic substance. FIG. 3B is a perspective view for illustrating a columnar scraper having a magnetic substance used in the central part and a non-magnetic substance in the peripheral part. FIG. 3C is a perspective view for illustrating a cylindrical scraper made of a magnetic substance. In FIG. 3A to FIG. 3C, the portion indicated by “m” indicates being a magnetic substance, the portion indicated by “n” indicates being a non-magnetic substance, and the portion indicated by “h” indicates being hollow.

### (Magnetic Field Applying Unit)

The magnetic field applying unit applies a magnetic field to the scraper. For the magnetic field applying unit, a magnet can be used, and a permanent magnet or an electromagnet can be used. As the permanent magnet, a ferrite magnet, a neodymium magnet, a samarium cobalt magnet, an alnico magnet, or the like can be used. When the permanent magnet is used as the magnetic field applying unit, there is a case in which a magnetic field is applied to the scraper by the magnetic field applying unit even at a time of culture that does not involve cell detachment. In such a case, the scraper is fixed at a time of cell culture, thereby facilitating reduction in damage caused to the cells.

### (Scraper Position Control Unit)

The scraper position control unit controls the position of the scraper by controlling the magnetic field applying unit. The scraper position control unit enables the scraper to slide on the culture surface. When the culture surface is a flat surface, it is preferred to move the magnetic field applying unit along the culture surface in a two-dimensional direction. When the scraper has a length enough to cover a width of the culture surface, a one-axis stage can be used as the scraper position control unit. Meanwhile, when a length of the scraper is shorter than the width of the culture surface, a two-axis stage is preferred to be used as the scraper position control unit.

### (Movement of Scraper)

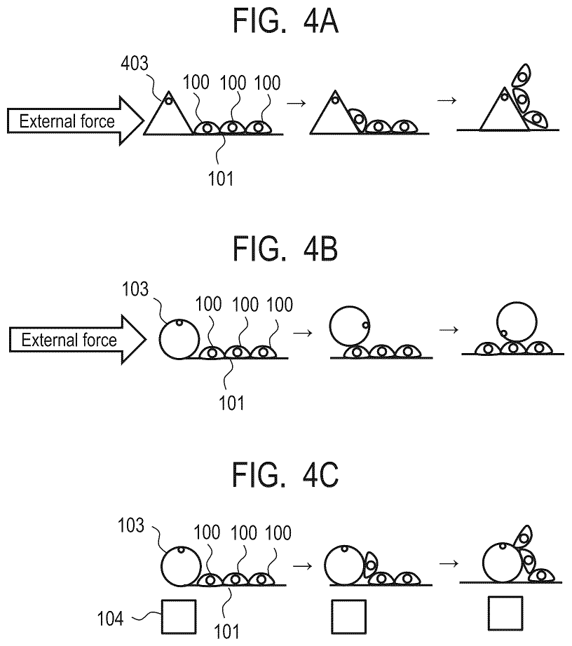

FIG. 4A to FIG. 4C are each an illustration of how a scraper is coming into contact with cells. Cells 100 are illustrated in FIG. 4A to FIG. 4C. When such a member as the scraper is installed without being fixed on a culture surface, the scraper moves on the culture surface due to an external force (for example, gravity or a water flow) at a time of culture medium replacement or the like during culture. FIG. 4A is a view for illustrating a state of the cells 100 exhibited when a related-art scraper 403 with an angular shape which does not rotate on the culture surface is moving on the culture surface 101 due to the external force under a state in which no magnetism is applied thereto. The scraper 403 takes a form of dragging on the culture surface 101, and is liable to detach the cells 100 at an unintended timing such as during a culture period. FIG. 4B is a view for illustrating a state of the cells 100 exhibited when the scraper 103 in the present invention is moving on the culture surface 101 under the state in which no magnetism is applied thereto. The scraper 103 moves on the culture surface 101 while rotating, and hence a frictional force is lowered, thereby being able to suppress an amount of the cells 100 that are unintentionally detached unlike in FIG. 4A.

FIG. 4C is a view for illustrating a state of the cells 100 exhibited when the scraper 103 in the present invention is moving on the culture surface 101 under a state in which magnetism is applied thereto. The scraper 103 is fixed in a magnetic pole direction by the magnetic field applying unit 104, and thereby takes a form of dragging on the culture surface 101 to enable the cells 100 to be detached.

In this manner, the scraper in the present invention moves on the culture surface while rotating under the state in which no magnetic field is applied thereto, and even when the scraper is installed on the culture surface, reduces damage to the cells during culture, while the scraper can detach cells by having magnetism being applied thereto at the time of detachment.

As a method of controlling the position of the scraper, it is possible to fix the magnetic field applying unit to the scraper position control unit, move the magnetic field applying unit by moving the scraper position control unit, and thereby move the scraper to which a magnetic field is applied.

### (Scraper Holding Portion and Scraper Moving Unit)

The cell detachment system according to the present invention is a cell detachment system including: a scraper having magnetism, for detaching cells adhering to a culture surface; a magnetic field applying unit that applies a magnetic field to the scraper; and a scraper position control unit that controls a position of the scraper by controlling the magnetic field applying unit, in which the scraper moves while rotating under a state in which no magnetic field is applied thereto, and under a state in which a magnetic field is applied thereto by the magnetic field applying unit, is caused to move, without rotating, by the scraper position control unit so as to detach the adhering cells, and can be provided as the cell detachment system further including a scraper holding portion that holds the scraper at a position out of contact with the culture surface.

Further, the cell detachment system according to the present invention may be a cell detachment system including: a scraper having magnetism, for detaching cells adhering to a culture surface; a magnetic field applying unit that applies a magnetic field to the scraper; a scraper position control unit that controls a position of the scraper by controlling the magnetic field applying unit; and a scraper holding portion that holds the scraper at a position out of contact with the culture surface.

Further, the cell detachment system according to the present invention may be a cell detachment system including a scraper moving unit that moves the scraper held by the scraper holding portion to the culture surface.

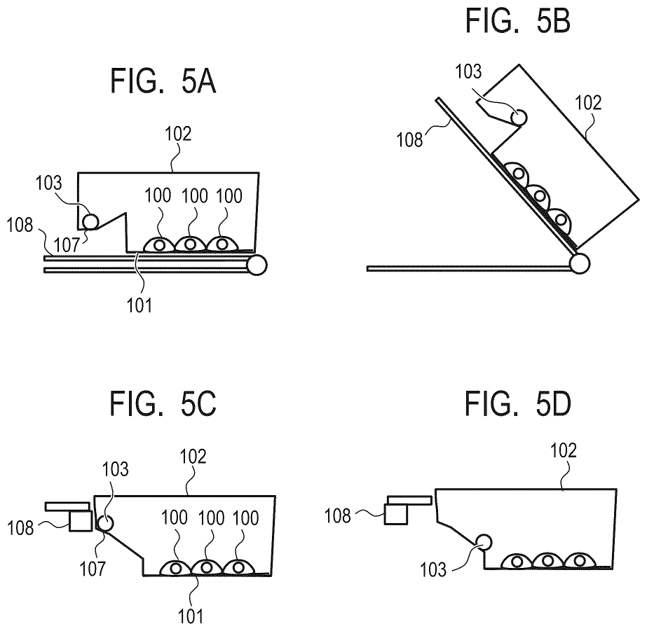

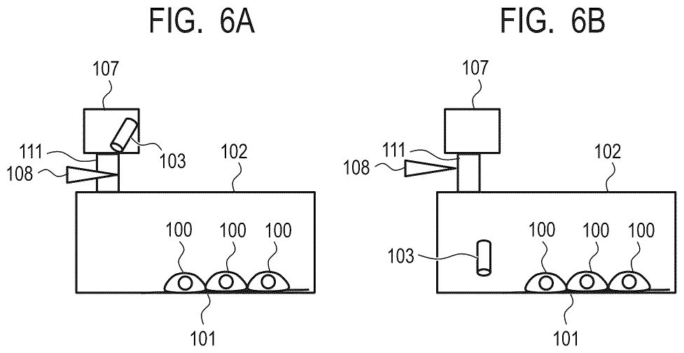

It is important to take suitable measures to prevent the scraper from damaging the cells being cultured. As such measures, it is effective to provide the scraper holding portion for holding the scraper at a position different from the culture surface during cell culture. Examples of a form of the scraper holding portion include a form of including the scraper holding portion inside the culture vessel and a form of including the scraper holding portion outside the culture vessel. As the form of including the scraper holding portion inside the culture vessel, for example, a space and a slope may be provided in the culture vessel to hold the scraper, or a magnet may be used to hold the scraper. Examples of the form of including the scraper holding portion outside the culture vessel include a form of providing a box-shaped scraper holding portion outside the culture vessel. When the cell detachment system includes the scraper holding portion, it is preferred to provide the scraper moving unit that moves the scraper held by the scraper holding portion to the culture surface. For example, a magnet may be used to provide the scraper moving unit, or a lid, a valve, or the like that can be opened and closed may be used to provide the scraper moving unit. The scraper holding portion and the scraper moving unit are described below with reference to FIG. 5A to FIG. 5D, FIG. 6A, and FIG. 6B. Forms of the scraper holding portion and the scraper moving unit are not limited to forms described below. Any forms can be used as long as such a scraper holding portion as to hold the scraper at a position out of contact with the culture surface is provided and the scraper can be moved to the culture surface by the scraper moving unit.

FIG. 5A to FIG. 5D are schematic views for each illustrating an example of the cell detachment system in which the scraper holding portion 107 is installed in the culture vessel 102. FIG. 5A is a view for illustrating a state during cell culture in the cell detachment system in which the scraper holding portion 107 has a slope. During culture of the cells 100, the scraper 103 is prevented from moving to the culture surface 101 by the scraper holding portion 107 having a slope. This also prevents the cells 100 being cultured from being detached. At a time of detachment, a scraper moving unit 108 is used to move the scraper 103 to the culture surface 101. As the scraper moving unit 108, a movable stage or the like can be used, and when the cell detachment system includes an image pickup apparatus, the scraper moving unit 108 is preferred to be arranged so as not to hinder the image pickup apparatus from picking up an image. FIG. 5B is a view for illustrating how to move the scraper 103 to the culture surface 101 in the above-mentioned cell detachment system after cell culture. In a case of this form, when the scraper 103 has a shape that allows the scraper 103 to move while rotating under the state in which no magnetic field is applied thereto, it is possible to move the scraper 103 to the culture surface 101 only by tilting the culture vessel 102 by moving the scraper moving unit 108.

In addition, a magnet can be used as the scraper moving unit 108. FIG. 5C is a view for illustrating a state during cell culture in the cell detachment system in which the scraper 103 is held by being attracted by a magnet. During culture, the scraper 103 is secured in the scraper holding portion 107 by being attracted by the scraper moving unit 108 that is a magnet. FIG. 5D is a schematic view for illustrating how to move the scraper 103 to the culture surface 101 in the above-mentioned cell detachment system after cell culture. At the time of cell detachment, it is possible to move the scraper 103 to the culture surface 101 by releasing the magnetic force of the scraper moving unit 108.

FIG. 6A and FIG. 6B are views for each illustrating an example of the cell detachment system in which the scraper holding portion 107 is installed outside the culture vessel 102. FIG. 6A is a schematic view for illustrating a state during cell culture in the above-mentioned cell detachment system. The culture vessel 102 and the scraper holding portion 107 that holds the scraper 103 are connected to each other through the tube 111. The above-mentioned configuration enables the scraper 103 to detach the cells while a closed system is maintained. The tube 111 is provided with a valve that serves as the scraper moving unit 108, and during culture, closing the scraper moving unit 108 can keep the scraper 103 in the scraper holding portion 107.

FIG. 6B is a schematic view for illustrating how to move the scraper 103 to the culture surface 101 after cell culture. During culture, as illustrated in FIG. 6A, the scraper 103 is secured in the scraper holding portion 107, thereby preventing the cells 100 from being detached during culture. At the time of detachment, it is possible to move the scraper 103 to the culture surface 101 by opening the scraper moving unit 108 to pass the scraper 103 through the inside of the tube 111. It is also possible to connect supply ports to the scraper holding portion 107 and to inject some sample solutions and the like through those ports. A method of moving the scraper 103 in the tube 111 can be achieved by flowing a culture medium, PBS, or another liquid together, or the scraper 103 can be moved therein simply by gravity. When the scraper 103 has a shape that allows the scraper 103 to rotate on a surface, the scraper 103 is less likely to get caught when passing through the inside of the tube 111.

### (Image Pickup Apparatus)

The cell detachment system according to the present invention can include an image pickup apparatus that acquires information regarding a state of the cells adhering to the culture surface. Examples of the image pickup apparatus include a CMOS camera and a CCD camera. Through use of the image pickup apparatus, it is possible to acquire information regarding the state of cells adhering to the culture surface, such as the areas of colonies included in the culture surface, and to determine whether or not a desired amount of cells have been cultured. It is also effective to incorporate processing in which an adhesion region of cells is examined from an image acquired by the image pickup apparatus and the scraper is moved to the adhesion region to detach cells in a desired region.

### (Information Processing Unit)

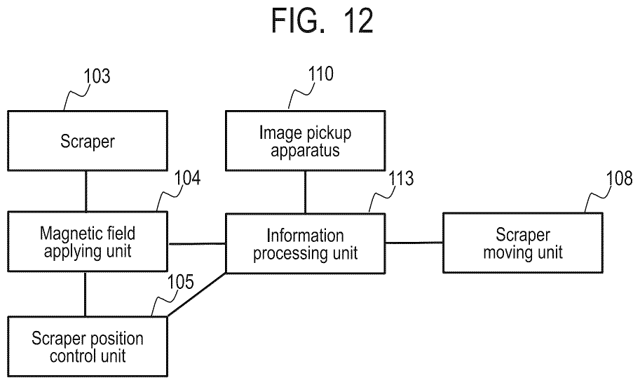

The cell detachment system according to the present invention can include an information processing unit. In particular, the cell detachment system according to the present invention can include an information processing unit configured to cause the scraper moving unit to move the scraper from the scraper holding portion to the culture surface based on the information acquired by the image pickup apparatus. FIG. 12 is a block diagram for illustrating a configuration example of the cell detachment system according to the present invention which includes the image pickup apparatus 110 and an information processing unit 113. The information processing unit 113 can process information regarding the state of cells adhering to the culture surface, in particular, for example, information regarding the state of cells adhering to the culture surface acquired by the image pickup apparatus 110. Further, for example, the information processing unit 113 can control the scraper position control unit 105 for moving the scraper 103 at the time of cell detachment. Further, for example, the information processing unit 113 can turn on and off a switch when the magnetic field applying unit 104 is an electromagnet or the like. Further, for example, the information processing unit 113 can issue an instruction to move the scraper 103 from the scraper holding portion to the culture surface through use of the scraper moving unit 108 at the time of cell detachment. For example, the above-mentioned instruction can be issued after it is determined whether or not a desired amount of cells have been cultured based on the areas of colonies included in the culture surface or other information acquired by the image pickup apparatus 110. The information processing unit 113 can also determine whether or not a desired amount of cells have been cultured based on, for example, information on the areas of colonies included in the culture surface which has been acquired by the image pickup apparatus 110, and issue an instruction to continue the culture or an instruction to end the culture and shift to the cell detachment. In a case of cells having a stable proliferation rate, the information processing unit 113 can also be used to set in advance the cell detachment to start after a lapse of a specified culture time period, and issue an instruction to perform the cell detachment after the lapse of the culture time period. In addition, for example, it is possible to issue an instruction to perform processing for detaching cells in a desired region by examining an adhesion region of cells from an image acquired by the image pickup apparatus 110 and moving the scraper 103 to the adhesion region through use of the scraper position control unit 105.

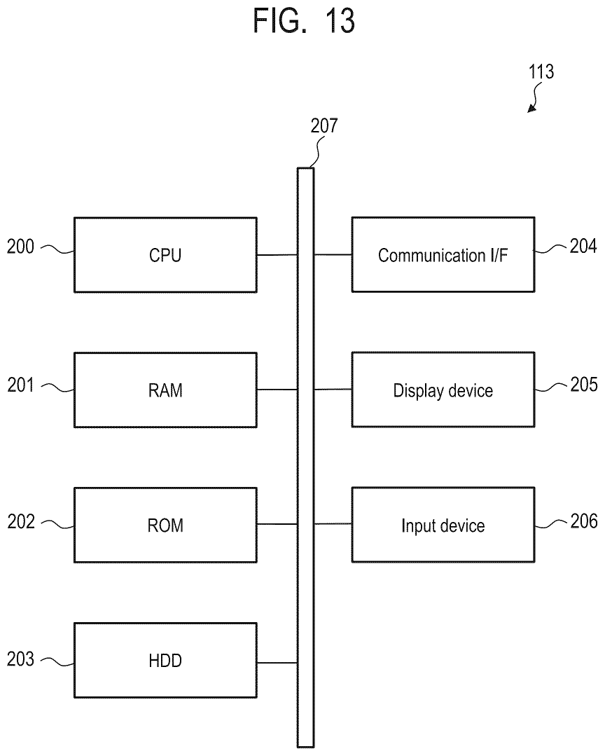

FIG. 13 is a block diagram for illustrating a hardware configuration example of the information processing unit 113 in the present invention. The information processing unit 113 has functions of a computer. For example, the information processing unit 113 may be configured unitarily with a desktop personal computer (PC), a laptop PC, a tablet PC, or a smartphone, for example. The information processing unit 113 includes, in order to implement functions as a computer that performs arithmetic operation and storage, a central processing unit (CPU) 200, a random-access memory (RAM) 201, a read-only memory (ROM) 202, and a hard disk drive (HDD) 203. The information processing unit 113 also includes a communication interface (I/F) 204, a display device 205, and an input device 206. The CPU 200, the RAM 201, the ROM 202, the HDD 203, the communication I/F 204, the display device 205, and the input device 206 are connected to each other via a bus 207. The display device 205 and the input device 206 may be connected to the bus 207 via a drive device (not shown) for driving those devices.

In FIG. 13, the various components forming the information processing unit 113 are illustrated as an integrated device, but some of the functions of those components may be implemented by an external device. For example, the display device 205 and the input device 206 may be external devices different from the components implementing the functions of the computer including the CPU 200, for example.

The CPU 200 performs predetermined operations in accordance with programs stored in, for example, the RAM 201 and the HDD 203, and also has a function of controlling each component of the information processing unit 113. The RAM 201 is built from a volatile storage medium, and provides a temporary memory area required for the operations of the CPU 200. The ROM 202 is built from a non-volatile storage medium, and stores required information such as programs to be used for the operations of the information processing unit 113. The HDD 203 is formed of a nonvolatile storage medium, and is a storage device that stores information regarding the state of cells and the like.

The communication I/F 204 is a communication interface based on a standard such as Wi-Fi (trademark) or 4G, and is a module for communicating to and from another device. The display device 205 is, for example, a liquid crystal display or an organic light emitting diode (OLED) display, and is used for displaying moving images, still images, and characters, for example. The input device 206 is, for example, a button, a touch panel, a keyboard, or a pointing device, and is used by a user to operate the information processing unit 113. The display device 205 and the input device 206 may be integrally formed as a touch panel.

The hardware configuration illustrated in FIG. 13 is an example, and devices other than the illustrated devices may be added, or some of the illustrated devices may be omitted. Further, some of the devices may be substituted with another device having the same function. Moreover, some of the functions may be provided by another device via a network, and the functions to be implemented may be shared and implemented by a plurality of devices. For example, the HDD 203 may be substituted with a solid state drive (SSD) which uses a semiconductor element, such as a flash memory, or may be substituted with cloud storage.

The CPU 200 implements various functions by loading a program stored in the ROM 202 or the like into the RAM 201 and executing the program. The CPU 200 also controls the display device 205. The CPU 200 also controls the HDD 203.

### (Cell Culture System)

The cell culture system according to the present invention is a cell culture system including: the cell detachment system of the present invention; and a culture vessel including the culture surface. Further, the cell culture system according to the present invention can include a cell culture unit. Examples of the cell culture unit include a heat retaining unit that maintains the entire cell culture system at 37° C., a unit that maintains the entire cell culture system under a CO₂ atmosphere, and a unit that maintains the entire cell culture system at a humidity of 95%. Specifically, an incubator in which the entire culture vessel is maintained at a temperature of 37° C., a CO₂ concentration of 5%, and a humidity of 95% is given as an example. In the following embodiments, cells are seeded and cultured by a supply and drainage port using a tube, but cells may be seeded through use of a pipette.

Through use of the above-mentioned system, it is possible to provide a cell detachment system that reduces physical damage caused to cells during cell culture as much as possible and also enables a scraper to detach cells while a closed system is maintained.

### First Embodiment

Now, a cell detachment system and a cell culture system including the cell detachment system according to a first embodiment are described. In the figures, like components are denoted by like reference numerals in principle, and description thereof is omitted.

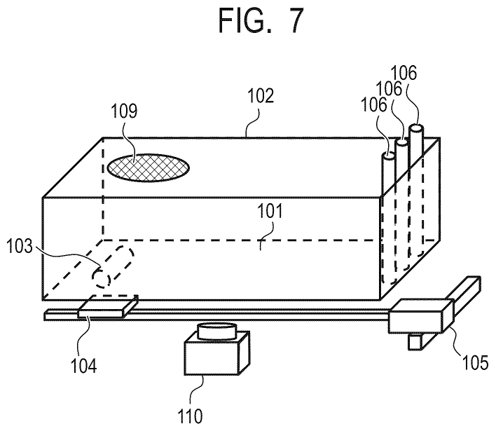

FIG. 7 is a schematic view for illustrating an example of an overall configuration of the cell culture system including the cell detachment system according to the first embodiment. In this system, a plurality of tubular supply and drainage ports 106 made of polystyrene are installed at an edge of the culture vessel 102 being a rectangular parallelepiped closed container made of polystyrene. In addition, a membrane filter 109 through which a gas such as CO₂ can pass is installed in the upper surface. The upper surface of the culture vessel 102 is kept closed except for a part in which the membrane filter 109 is installed and parts in which the supply and drainage ports 106 are installed, and is connected to the outside through the supply and drainage ports 106. Further, a lower surface of the culture vessel 102 on an inner side thereof is the culture surface 101, and is subjected to a surface treatment for allowing cells to adhere thereto. In the culture vessel 102, a columnar SUS440-made metal rod having a diameter of 1 mm and a length of 5 mm is installed as the scraper 103. Below the culture vessel 102, an electromagnet is arranged as the magnetic field applying unit 104, and the magnetic field applying unit 104 is installed in the scraper position control unit 105 being a two-axis automatic stage. In addition, in a central part below the culture vessel 102, a CMOS camera is installed as the image pickup apparatus 110 that allows observation of the culture surface 101.

A procedure of actual cell culture and detachment using this system is described below.

### (Initial Setting)

Three supply and drainage ports 106 are prepared, and a silicone tube (not shown) is attached to each thereof.

The first supply and drainage port 106 can be connected to a container (not shown) for a culture medium, a container (not shown) for an enzyme solution at the time of detachment, a container (not shown) for PBS, and a container (not shown) for a cell suspension in which cells before culture are mixed. The second supply and drainage port 106 can be connected to a waste container (not shown) for accumulating waste liquids. The third supply and drainage port 106 can be connected to a collection container (not shown) for accumulating the cell suspension including detached cells. The silicone tube is provided with a joint (not shown) for connection to each container. The silicone tube to be connected to the first supply and drainage port 106 is branched off so as to correspond to types of the sample solutions and the like, and opening and closing of those branches can be switched by valves. A sterilization treatment is performed with gamma rays on the entire culture vessel 102 to which those silicone tubes are connected.

In a case of starting cell culture, the respective silicone tubes are connected to the respective containers containing the sample solutions and the like, the waste container, and the collection container. In this case, those are required to be aseptically connected. After the connection, the respective tubes are connected to tube pumps (not shown).

Feeding of the liquids from the respective containers of the sample solutions and the like to the culture vessel 102 and feeding of the liquids from the culture vessel 102 to the waste container and the collection container are carried out by the respective tube pumps. As an actuator for feeding the liquids, the tube pump has been used in the first embodiment, but in place of the tube pump, any actuator can be used as long as the actuator can achieve a closed system. Lastly, the entire cell culture system except for the respective containers of the sample solutions and the like is installed in an incubator (not shown) having the inside maintained at a temperature of 37° C., a CO₂ concentration of 5%, and a humidity of 95%.

### (Cell Culture)

First, a cell suspension being a mixed solution of a culture medium, extracellular matrices, and cells to be cultured is fed to the culture vessel 102 through the supply and drainage port 106. The cell suspension is fed by an amount enough to immerse the cell suspension over an entire area of the culture surface 101. After that, the culture vessel 102 is kept stored in the incubator for a certain period of time. The culture medium replacement is sometimes carried out, and in that case, after the culture medium in the culture vessel 102 is moved to the waste container, a new culture medium is moved into the culture vessel. A frequency of this culture medium replacement is preferred to be set in accordance with the cells to be cultured. The culture medium replacement and the storage in the incubator are continued until the cells have been proliferated by a desired amount. Whether or not a desired amount of cells have been cultured can be determined from the areas of colonies included in the culture surface through use of the image pickup apparatus 110. This determination can also be made automatically by providing an information processing unit (not shown). A CMOS camera is used as the image pickup apparatus 110, but a CCD camera can be used as well.

### (Cell Detachment)

After a desired amount of cells have been cultured, the cells are detached. A timing for the cell detachment is determined from an image of the cells adhering to the culture surface 101 which has been acquired by the image pickup apparatus 110. It is effective to set, as the timing for the detachment, a timing at which the areas of colonies included in the culture surface have become equal to or larger than a predetermined area. This determination can also be made automatically by providing an information processing unit (not shown), and an instruction to shift to the cell detachment can also be issued. In the case of cells having a stable proliferation rate, the specified culture time period can be used to set as the timing for the detachment. In the case of providing the information processing unit, it is also possible to perform such a setting as to automatically shift to the cell detachment after the lapse of the specified culture time period.

First, after the culture medium in the culture vessel 102 is moved to the waste container through the supply and drainage port 106, PBS is fed into the culture vessel 102 through the supply and drainage port 106 until the culture surface 101 is immersed. Then, after the PBS in the culture vessel 102 is moved to the waste container through the supply and drainage port 106, an enzyme solution such as trypsin is fed into the culture vessel 102 through the supply and drainage port 106 until the culture surface 101 is immersed, with the culture vessel 102 being maintained in the incubator at 37° C. for a certain period of time. After that, the enzyme solution in the culture vessel 102 is moved to the waste container through the supply and drainage port 106, and then the culture medium is fed into the culture vessel 102 through the supply and drainage port 106 until the culture surface 101 is immersed.

After that, the magnetic field applying unit 104 being an electromagnet is turned on, and is driven by the scraper position control unit 105 being a two-dimensional stage so that the magnetic field applying unit 104 passes through the entire area below the culture surface 101 two times. The scraper 103 can freely move on the culture surface 101 before culture is started, and hence the scraper 103 is not always present in a vicinity of the magnetic field applying unit 104 when the culture is started, but at a time of the first passage, the scraper 103 is reliably secured by the magnetic force of the magnetic field applying unit 104. For that reason, the two times of passage ensure that the scraper 103 passes through the entire area of the culture surface 101 at least once, to thereby be able to detach almost all the cells adhering to the culture surface 101.

It is also effective to incorporate processing in which an adhesion region of cells is examined from an image acquired by the image pickup apparatus 110 and the scraper 103 is moved to the adhesion region to detach cells in a desired region.

This processing may be manually performed, or may be automatically performed through use of the information processing unit.

### (Cell Collection)

In the culture vessel 102, the suspension of the culture medium and the detached cells is present. The tube pump is operated to move the cell suspension to the collection container through the supply and drainage port 106. In this case, it is possible to collect the cell suspension more efficiently by tilting the culture vessel 102 so that a side on which the supply and drainage ports 106 are installed becomes lower, which is accordingly effective.

As described above, according to the first embodiment, it is possible to provide a cell detachment system including: a scraper having magnetism, for detaching cells adhering to a culture surface; a magnetic field applying unit that applies a magnetic field to the scraper; and a scraper position control unit that controls a position of the scraper by controlling the magnetic field applying unit, in which the scraper moves while rotating under a state in which no magnetic field is applied thereto, and under a state in which a magnetic field is applied thereto by the magnetic field applying unit, is caused to move, without rotating, by the scraper position control unit so as to detach the adhering cells, and it is also possible to provide a cell culture system including the above-mentioned cell detachment system.

The first embodiment has been verified through use of iPS cells, but most types of cell can be used as long as the cells are adherent cells. In that case, types of culture medium and reagent and a timing of the detachment may be changed as appropriate in accordance with characteristics of each cell and a protocol to be implemented.

### Second Embodiment

Now, a cell detachment system and a cell culture system including the cell detachment system according to a second embodiment are described. In the figures, like components are denoted by like reference numerals in principle, and description thereof is omitted.

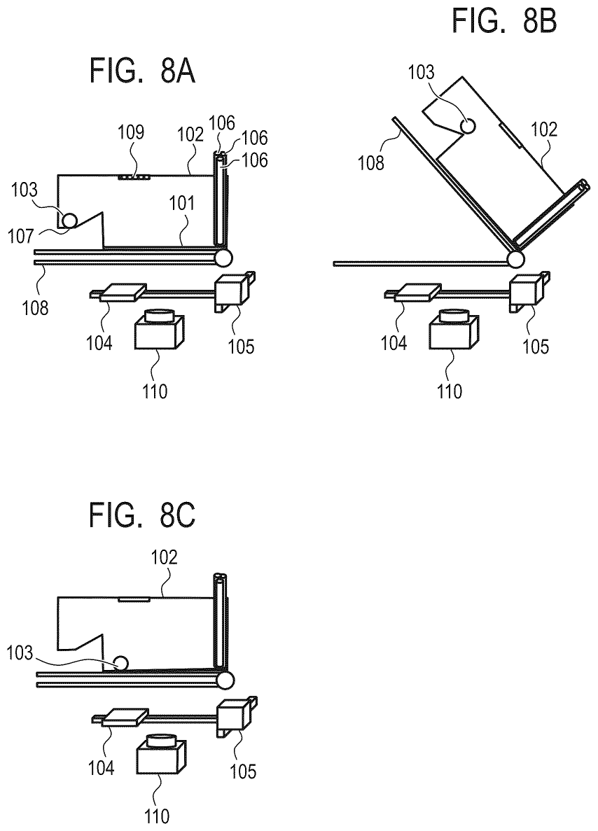

FIG. 8A to FIG. 8C are schematic views for each illustrating an example of an overall configuration of the cell culture system including the cell detachment system according to the second embodiment. FIG. 8A is a view for illustrating a state exhibited before the scraper 103 is moved to the culture surface 101.

In this system, the plurality of tubular supply and drainage ports 106 made of polystyrene are installed at an edge of the culture vessel 102 being a closed container made of polystyrene. In addition, the membrane filter 109 through which a gas such as CO₂ can pass is installed in the upper surface. Further, the lower surface of the culture vessel 102 on the inner side thereof is the culture surface 101, and is subjected to a surface treatment for allowing cells to adhere thereto. In the culture vessel 102, the scraper holding portion 107 provided with such a sloped wall as to prevent the scraper 103 from easily moving to the culture surface 101 is provided at a position out of contact with the culture surface 101.

In the scraper holding portion 107 in the culture vessel 102, a columnar SUS440-made metal rod having a diameter of 1 mm and a length of 5 mm is installed as the scraper 103. On the outside of the culture vessel 102, the magnetic field applying unit 104 being an electromagnet is arranged, and the magnetic field applying unit 104 is installed in the scraper position control unit 105 being a two-axis automatic stage. In addition, in a central part below the culture surface 101 of the culture vessel 102, a CMOS camera is installed as the image pickup apparatus 110 that allows observation of the culture surface 101. Below the culture vessel 102, the scraper moving unit 108 being a movable stage is installed.

The steps of the sections “(Initial Setting),” “(Cell Culture),” and “(Cell Collection)” in this system are the same as those in the first embodiment, hence description thereof is omitted, and only the step of the section “(Cell Detachment)” is described below.

### (Cell Detachment)

After a desired amount of cells have been cultured, the cells are detached. A timing for the cell detachment is determined from an image of the cells adhering to the culture surface 101 which has been acquired by the image pickup apparatus 110. It is effective to set, as the timing for the detachment, a timing at which the areas of colonies included in the culture surface have become equal to or larger than a predetermined area. This determination can also be made automatically by providing an information processing unit (not shown), and an instruction to shift to the cell detachment can also be issued. In the case of cells having a stable proliferation rate, the specified culture time period can be used to set as the timing for the detachment. In the case of providing the information processing unit, it is also possible to perform such a setting as to automatically shift to the cell detachment after the lapse of the specified culture time period.

First, after the culture medium in the culture vessel 102 is moved to the waste container through the supply and drainage port 106, PBS is fed into the culture vessel 102 through the supply and drainage port 106 until the culture surface 101 is immersed. Then, after the PBS in the culture vessel 102 is moved to the waste container through the supply and drainage port 106, an enzyme solution such as trypsin is fed into the culture vessel 102 through the supply and drainage port 106 until the culture surface 101 is immersed, with the culture vessel 102 being maintained in the incubator at 37° C. for a certain period of time. After that, the enzyme solution in the culture vessel 102 is moved to the waste container through the supply and drainage port 106, and then the culture medium is fed into the culture vessel 102 through the supply and drainage port 106 until the culture surface 101 is immersed.

Then, the scraper moving unit 108 is driven to tilt the culture vessel 102 as illustrated in FIG. 8B, to thereby move the scraper 103 to the culture surface 101 side, and then returns the culture vessel 102 to a horizontal position as illustrated in FIG. 8C. The driving of the scraper moving unit 108 may be manually performed, or can be automatically performed through use of the information processing unit.

After that, in the same manner as in the first embodiment, the magnetic field applying unit 104 being an electromagnet is turned on, and is driven by the driving of the scraper position control unit 105 being a two-dimensional stage so that the magnetic field applying unit 104 passes through the entire area below the culture surface 101 two times. In the same manner as in the first embodiment, at the time of the first passage, the scraper 103 is reliably secured by the magnetic force of the electromagnet. For that reason, the two times of passage of the magnetic field applying unit 104 ensure that the scraper 103 passes through the entire area of the culture surface 101 at least once, to thereby be able to detach almost all the cells adhering to the culture surface 101.

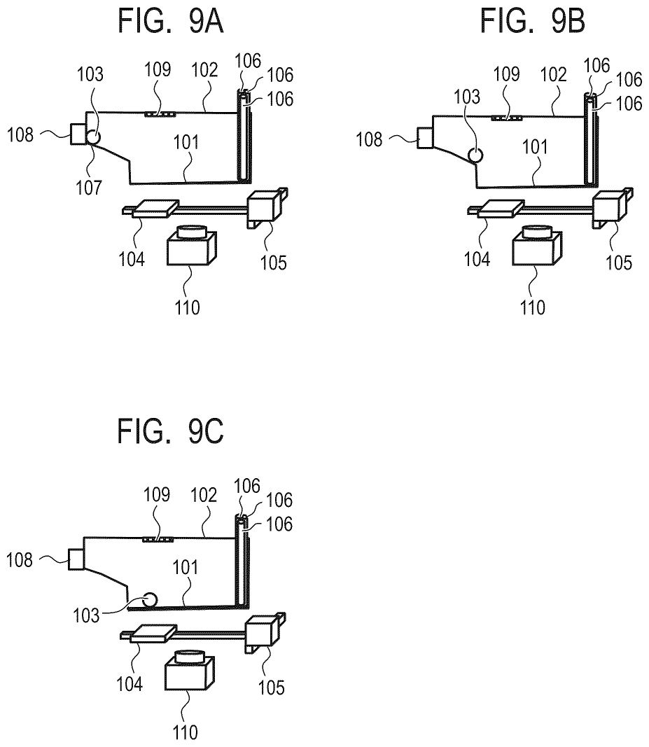

In another example, as illustrated in FIG. 9A to FIG. 9C, it is possible to provide a cell culture system including a cell detachment system that uses an electromagnet as the scraper moving unit 108. FIG. 9A to FIG. 9C are schematic views for each illustrating an example of an overall configuration of the cell culture system including the cell detachment system according to the second embodiment. FIG. 9A is a view for illustrating a state exhibited before the scraper 103 is moved to the culture surface 101.

In this system, the plurality of tubular supply and drainage ports 106 made of polystyrene are installed at an edge of the culture vessel 102 being a closed container made of polystyrene. In addition, the membrane filter 109 through which a gas such as CO₂ can pass is installed in the upper surface. Further, the lower surface of the culture vessel 102 on the inner side thereof is the culture surface 101, and is subjected to a surface treatment for allowing cells to adhere thereto. In the culture vessel 102, the scraper holding portion 107 including a sloped wall and holding the scraper 103 is provided at a position out of contact with the culture surface 101, and the scraper moving unit 108 being an electromagnet is provided in a vicinity of the scraper holding portion 107.

In the culture vessel 102, a columnar SUS440-made metal rod having a diameter of 1 mm and a length of 5 mm is installed in the scraper holding portion 107 as the scraper 103. On the outside of the culture vessel 102, the magnetic field applying unit 104 being an electromagnet is arranged, and the magnetic field applying unit 104 is installed in the scraper position control unit 105 being a two-axis automatic stage. In addition, in the central part below the culture surface 101 of the culture vessel 102, a CMOS camera is installed as the image pickup apparatus 110 that allows observation of the culture surface 101.

The configuration illustrated in FIG. 9A is the same as the configuration illustrated in FIG. 8A except that an electromagnet is installed as the scraper moving unit 108 in the vicinity of the scraper holding portion 107 provided with a slope directed toward the culture surface 101 and the scraper 103 is held by being attracted to a wall surface of the scraper holding portion 107 by a magnetic force.

FIG. 9B is a view for illustrating how to move the scraper 103 to the culture surface 101. FIG. 9C is a view for illustrating a state in which the movement of the scraper 103 to the culture surface 101 has been completed. When the scraper moving unit 108 is turned off, the scraper 103 moves from the sloped surface of the scraper holding portion 107 to the culture surface 101. Then, the scraper 103 is moved by the magnetic field applying unit 104 being moved by the scraper position control unit 105 in accordance with the same procedure as in the configuration illustrated in FIG. 8C, to thereby be able to detach the cells. The turning on and off of the switch of the scraper moving unit 108 may be manually performed, or can be automatically performed through use of the information processing unit.

As described above, in the second embodiment, the scraper holding portion is provided at a position out of contact with the culture surface in the culture vessel, and the scraper moving unit that moves the scraper held by the scraper holding portion to the culture surface is provided. Accordingly, it is possible to provide a cell detachment system that reduces physical damage caused to cells during cell culture and also enables cells to be detached while a closed system is maintained, and it is also possible to provide a cell culture system including the above-mentioned cell detachment system. Further, at this time, when the scraper can move while rotating under the state in which no magnetic field is applied thereto, the scraper can easily move to the culture surface.

The second embodiment has been verified through use of iPS cells, but most types of cell can be used as long as the cells are adherent cells. In that case, types of culture medium and reagent and a timing of the detachment may be changed as appropriate in accordance with characteristics of each cell and a protocol to be implemented.

### Third Embodiment

Now, a cell detachment system and a cell culture system including the cell detachment system according to a third embodiment are described. In the figures, like components are denoted by like reference numerals in principle, and description thereof is omitted.

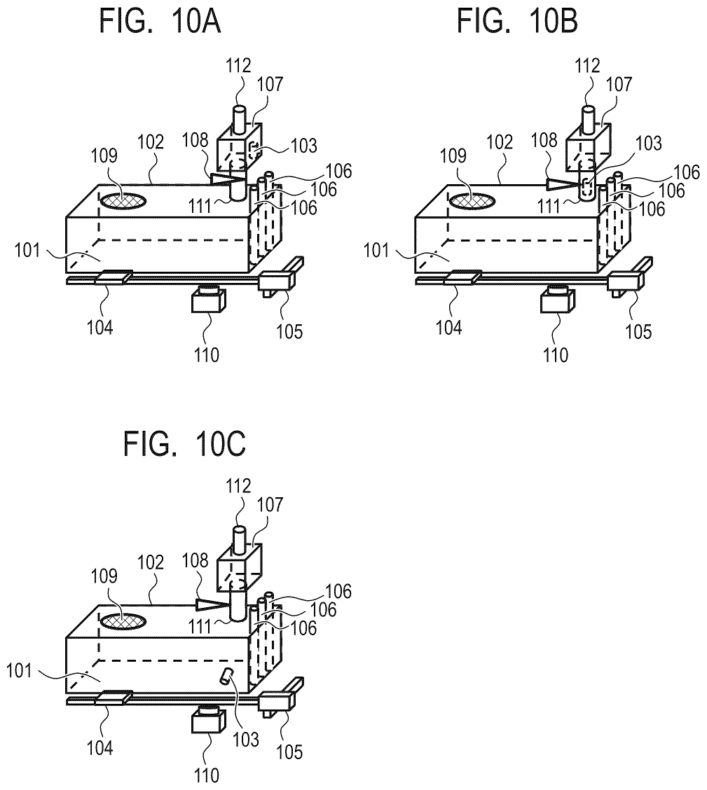

FIG. 10A to FIG. 10C are schematic views for each illustrating an example of an overall configuration of the cell culture system including the cell detachment system according to the third embodiment. FIG. 10A is a view for illustrating a state exhibited before the scraper 103 is moved to the culture surface 101.

In this system, the plurality of tubular supply and drainage ports 106 made of polystyrene are installed at an edge of the culture vessel 102 being a closed container made of polystyrene. In addition, the membrane filter 109 through which a gas such as CO₂ can pass is installed in the upper surface. Further, the lower surface of the culture vessel 102 on the inner side thereof is the culture surface 101, and is subjected to a surface treatment for allowing cells to adhere thereto. On the outside of the culture vessel 102, the scraper holding portion 107 made of polystyrene and connected to the culture vessel 102 through the tube 111 is prepared. The scraper holding portion 107 is further provided with a supply port 112, and can be connected to each of the containers of the sample solutions and the like through the silicone tube. The tube 111 is provided with a valve as the scraper moving unit 108. In the scraper holding portion 107, a columnar SUS440-made metal rod having a diameter of 1 mm and a length of 5 mm is installed as the scraper 103. On the outside of the culture vessel 102, the magnetic field applying unit 104 being an electromagnet is arranged, and the magnetic field applying unit 104 is installed in the scraper position control unit 105 being a two-axis automatic stage. In addition, in the central part below the culture surface 101 of the culture vessel 102, a CMOS camera is installed as the image pickup apparatus 110 that allows observation of the culture surface 101.

The steps of the sections “(Initial Setting),” “(Cell Culture),” and “(Cell Collection)” in this system are the same as those in the first embodiment, hence description thereof is omitted, and only the step of the section “(Cell Detachment)” is described below.

### (Cell Detachment)

After it is confirmed that a desired amount of cells have been cultured, the cells are detached. A timing for the cell detachment is determined from an image of the cells adhering to the culture surface 101 which has been acquired by the image pickup apparatus 110. It is effective to set, as the timing for the detachment, a timing at which the areas of colonies included in the culture surface have become equal to or larger than a predetermined area. This determination can also be made automatically by providing an information processing unit (not shown), and an instruction to shift to the cell detachment can also be issued. In the case of cells having a stable proliferation rate, the specified culture time period can be used to set as the timing for the detachment. In the case of providing the information processing unit, it is also possible to perform such a setting as to automatically shift to the cell detachment after the lapse of the specified culture time period.

First, after the culture medium in the culture vessel 102 is moved to the waste container through the supply and drainage port 106, PBS is fed into the culture vessel 102 through the supply and drainage port 106 until the culture surface 101 is immersed. Then, after the PBS in the culture vessel 102 is moved to the waste container through the supply and drainage port 106, an enzyme solution such as trypsin is fed into the culture vessel 102 through the supply and drainage port 106 until the culture surface 101 is immersed, with the culture vessel 102 being maintained in the incubator at 37° C. for a certain period of time. After that, the enzyme solution in the culture vessel 102 is moved to the waste container through the supply and drainage port 106, and then the culture medium is fed into the culture vessel 102 through the supply and drainage port 106 until the culture surface 101 is immersed.

Then, the valve serving as the scraper moving unit 108 is released, and the scraper 103 is moved to the culture surface 101 by being passed through the inside of the tube 111. FIG. 10B is a view for illustrating how to move the scraper to the culture surface. The driving of the scraper moving unit 108 may be manually performed, or can be automatically performed through use of the information processing unit. FIG. 10C is a view for illustrating a state in which the movement of the scraper to the culture surface has been completed. In a case of moving the scraper 103, it is also effective to inject a culture medium to the scraper holding portion 107 through the supply port 112, release the scraper moving unit 108, and then move the scraper 103 with a force of flowing the culture medium.

After that, in the same manner as in the first embodiment, the magnetic field applying unit 104 being an electromagnet is turned on, and is driven by the driving of the scraper position control unit 105 being a two-dimensional stage so that the magnetic field applying unit 104 passes through the entire area below the culture surface 101 two times. In the same manner as in the first embodiment, at the time of the first passage, the scraper 103 is reliably secured by the magnetic force of the electromagnet. For that reason, the two times of passage of the magnetic field applying unit 104 ensure that the scraper 103 passes through the entire area of the culture surface 101 at least once, to thereby be able to detach almost all the cells adhering to the culture surface 101.

As described above, in the third embodiment, the scraper holding portion, and the scraper moving unit that moves the scraper held by the scraper holding portion to the culture surface are provided on the outside of the culture vessel. Accordingly, it is possible to provide a cell detachment system that reduces physical damage caused to cells during cell culture and also enables cells to be detached while a closed system is maintained, and it is also possible to provide a cell culture system including the above-mentioned cell detachment system. Further, at this time, when the scraper can move while rotating under the state in which no magnetic field is applied thereto, the scraper is less likely to get caught in the tube, and can smoothly pass therethrough.

The third embodiment has been verified through use of iPS cells, but most types of cell can be used as long as the cells are adherent cells. In that case, types of culture medium and reagent and a timing of the detachment may be changed as appropriate in accordance with characteristics of each cell and a protocol to be implemented.

### Fourth Embodiment

Now, a cell detachment system and a cell culture system including the cell detachment system according to a fourth embodiment are described. In the figures, like components are denoted by like reference numerals in principle, and description thereof is omitted.

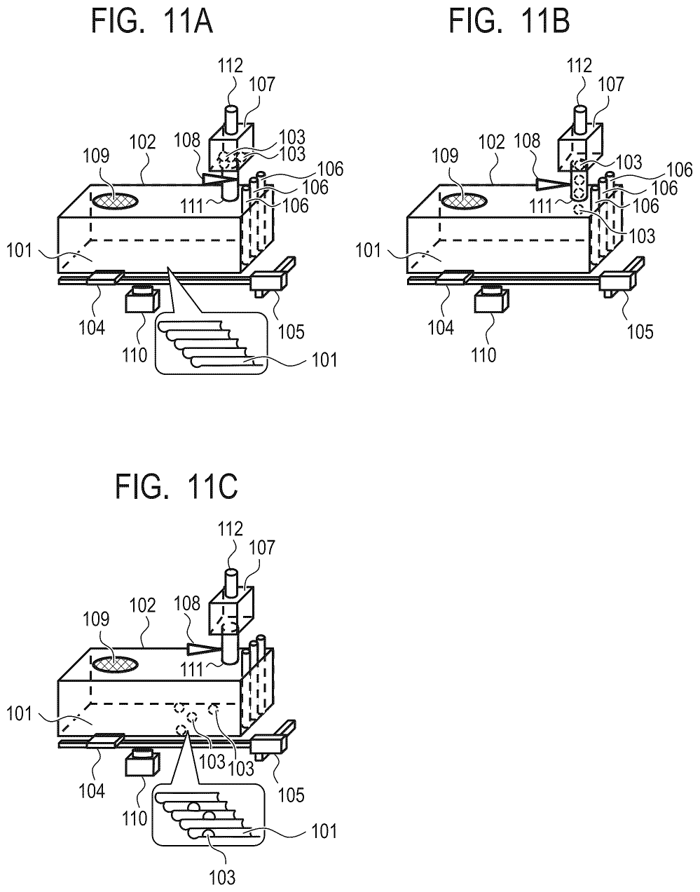

FIG. 11A to FIG. 11C are schematic views for each illustrating an example of an overall configuration of the cell culture system including the cell detachment system according to the fourth embodiment. FIG. 11A is a view for illustrating a state exhibited before scrapers 103 are moved to the culture surface 101. FIG. 11B is a view for illustrating how to move the scrapers 103 to the culture surface 101. FIG. 11C is a view for illustrating a state in which the movement of the scrapers 103 to the culture surface 101 has been completed. Figures in the balloons of FIG. 11A and FIG. 11C are detailed views for each illustrating how the culture surface 101 and the scrapers 103 installed on the culture surface 101 appear.

This system is almost the same as the system in the third embodiment, and differs in that a plurality of rows of recessed curved surface substrates are arranged as the culture surface 101 and that the scrapers 103 each having a spherical shape are used.

The culture surface 101 has a form in which the recessed curved surface substrates each having a diameter of φ1 mm are arranged in parallel to each other, and is formed as a part of the culture vessel 102 made of polystyrene.

As each of the scrapers 103, an SUS440-made spherical body having a diameter of φ1 mm is used. At the time of detachment, when a magnetic field is applied to the scraper 103, the scraper 103 is brought into tightly close contact with the culture surface 101, and the scraper 103 is moved under that state, to thereby be able to scrape off the cells adhering to the culture surface 101.

The steps of the sections “(Initial Setting),” “(Cell Culture),” “(Cell Collection),” and “(Cell Detachment)” in this system are the same as those in the third embodiment, and hence description thereof is omitted.

As described above, in the fourth embodiment, the scraper holding portion, and the scraper moving unit that moves the scraper held by the scraper holding portion to the culture surface are provided on the outside of the culture vessel. In addition, the substrates each having a recessed curved shape are installed as the culture surface, and the scrapers each having a spherical shape are provided. Accordingly, it is possible to provide a cell detachment system that reduces physical damage caused to cells during cell culture and also enables cells to be detached while a closed system is maintained, and it is also possible to provide a cell culture system including the above-mentioned cell detachment system. Further, each scraper has a spherical shape, and is thus easy to move in the tube from the scraper holding portion to the culture surface.

The fourth embodiment has been verified through use of iPS cells, but most types of cell can be used as long as the cells are adherent cells. In that case, types of culture medium and reagent and a timing of the detachment may be changed as appropriate in accordance with characteristics of each cell and a protocol to be implemented.

It is possible to provide the cell detachment system that reduces physical damage caused to cells during cell culture and also enables the scraper to detach cells while a closed system is maintained.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2023-098040, filed Jun. 14, 2023, which is hereby incorporated by reference herein in its entirety.

What is claimed is:

1. A cell detachment system comprising:
a scraper having magnetism, for detaching cells adhering to a culture surface;
a magnetic field applying unit configured to apply a magnetic field to the scraper; and
a scraper position control unit configured to control a position of the scraper by controlling the magnetic field applying unit,
wherein the scraper is configured to move while rotating under a state in which no magnetic field is applied thereto, and under a state in which a magnetic field is applied thereto by the magnetic field applying unit, to be caused to move, without rotating, by the scraper position control unit so as to detach the adhering cells.

2. The cell detachment system according to claim 1, wherein a shape of the scraper comprises one of a spherical shape, an ellipsoidal shape, a columnar shape, or a cylindrical shape.

3. The cell detachment system according to claim 1, further comprising a scraper holding portion configured to hold the scraper at a position out of contact with the culture surface.

4. The cell detachment system according to claim 3, further comprising a scraper moving unit configured to move the scraper held by the scraper holding portion to the culture surface.

5. The cell detachment system according to claim 4, further comprising an image pickup apparatus configured to acquire information regarding a state of the cells adhering to the culture surface.

6. The cell detachment system according to claim 5, further comprising an information processing unit configured to cause the scraper moving unit to move the scraper from the scraper holding portion to the culture surface based on the information acquired by the image pickup apparatus.

7. The cell detachment system according to claim 1, wherein the culture surface includes a recessed curved surface, and a shape of the scraper comprises one of a spherical shape or an ellipsoidal shape.

8. The cell detachment system according to claim 1, wherein the cells comprise adherent cells.

9. A cell culture system comprising:
the cell detachment system according to claim 1; and
a culture vessel including the culture surface.

10. The cell culture system according to claim 9, further comprising a cell culture unit.

11. A cell detachment system comprising:
a scraper having magnetism, for detaching cells adhering to a culture surface;
a magnetic field applying unit configured to apply a magnetic field to the scraper;
a scraper position control unit configured to control a position of the scraper by controlling the magnetic field applying unit; and
a scraper holding portion configured to hold the scraper at a position out of contact with the culture surface.

12. The cell detachment system according to claim 11, further comprising a scraper moving unit configured to move the scraper held by the scraper holding portion to the culture surface.

13. The cell detachment system according to claim 12, further comprising an image pickup apparatus configured to acquire information regarding a state of the cells adhering to the culture surface.

14. The cell detachment system according to claim 13, further comprising an information processing unit configured to cause the scraper moving unit to move the scraper from the scraper holding portion to the culture surface based on the information acquired by the image pickup apparatus.

15. The cell detachment system according to claim 11, wherein the cells comprise adherent cells.

16. A cell culture system comprising:
the cell detachment system according to claim 11; and
a culture vessel including the culture surface.

17. The cell culture system according to claim 16, further comprising a cell culture unit.
